(19) United States
(12) Patent Application Publication
(10) Pub. No.: US 2018/0147172 A1
(43) Pub. Date: May 31, 2018

(54) Compositions for Mitigating Head Trauma

(71) Applicant: Garrett E. Wdowin, Corona del Mar, CA (US)

(72) Inventor: Garrett E. Wdowin, Corona del Mar, CA (US)

(21) Appl. No.: 15/878,288

(22) Filed: Jan. 23, 2018

Related U.S. Application Data
(63) Continuation-in-part of application No. 15/276,654, filed on Sep. 26, 2016.

(51) Int. Cl. A61K 31/202; A61K 31/201; A61K 31/05; A61K 31/12

(52) U.S. Cl. CPC A61K 31/202; A61K 31/12; A61K 31/05; A61K 31/201

(57) ABSTRACT

A method for mitigating the effects of traumatic brain injury in an individual comprising is disclosed. The method includes administering an amount of a composition prior to a period wherein the traumatic brain injury is expected to occur, wherein the composition is comprised of: 300 mg and 500 milligrams (mg) of Docosahexaenoic acid (DHA), 50 mg/kg to 400 mg/kg (milligrams of composition per kilogram of body weight of the individual) of curcumin, and 10 mg to 100 mg of resveratrol. In some embodiments, the composition may include 350 mg of DHA, 150 mg/kg of curcumin and 30 mg of resveratrol. In additional embodiments, the composition may further include α-linoleic acid of an amount within the range of, for example, 1-2 g. The composition may be administered to the individual within a predetermined amount of time prior to a period wherein the traumatic brain injury is expected to occur.

### CROSS-REFERENCE TO RELATED APPLICATIONS

This continuation-in-part application claims the benefit of U.S. patent application Ser. No. 15/276,554, filed Sep. 26, 2016, the entire contents of which are incorporated by reference herein.

### FIELD OF THE INVENTION

Embodiments of the disclosure relate to the field of the prevention, mitigation and/or neurological damage associated with head trauma injuries. More particularly, one embodiment of the disclosure relates to a composition for preventing and/or mitigating neurological damage having the form of an edible gel to be taken prior to an event or activity having a high likelihood of resulting in an injury associated with head trauma.

### BACKGROUND

According to the Mayo Clinic, a traumatic brain injury may occur when an external mechanical force causes brain dysfunction (e.g., a violent blow or jolt to the head or body). The Mayo Clinic has stated that the severity of the injury may be influenced by one or more factors including the area of the head directly impacted, whether the brain moved back and forth in the skull upon impact, whether cellular structures were torn as a result of a severe rotational or spinning jolt, whether an object penetrated the skull, whether bleeding in or around the brain occurred, etc. Certain types of events are known to be likely to result in, or expected to cause, traumatic brain injury such as falls, collisions, fights or abuse, sports injuries and explosive blasts, among others.

In particular, a traumatic brain injury may cause bruising, torn tissues in the brain, bleeding, long-term complications, or even death. Traumatic brain injury may be mild, moderate or severe, with symptoms dependent on the severity of the trauma. For example, the Mayo Clinic has detailed that a mild traumatic brain injury may cause the injured person to experience physical symptoms such as loss of consciousness or nausea. Additional symptoms have also been detailed for traumatic brain injuries. With moderate or severe traumatic brain injury, an injured person may fall into a coma or a vegetative state, or experience seizures, fluid buildup in the cerebral ventricles and/or blood vessel damage. According to the Mayo Clinic, these symptoms may lead to additional complications including nerve damage, cognitive problems, issues with problem-solving and organization skills as well as social and behavioral problems.

One well-known symptom of traumatic brain injury is a concussion. Concussion symptoms may be immediately apparent or slow-to-develop, and according to the Mayo Clinic, include headache, loss of memory (amnesia) and confusion. Further, it is noted by the Mayo Clinic that suffering a concussion may change the levels of brain chemicals, which typically stabilize over time as the injured person recovers.

Current mechanisms for preventing or mitigating traumatic brain injury include physical measures such as buckling a seatbelt while riding in an automobile, cleaning spills so as to avoid slipping, and wearing proper protective equipment when participating in sporting events. However, there is a lack of compositions, e.g., a plurality of ingredients combined in one or more delivery mechanisms, that can be taken prior to particular events during which a concussion, or other traumatic brain injury, is expected or likely to occur.

### SUMMARY OF THE INVENTION

In general, embodiments of the disclosure describe compositions and methods for preventing or mitigating the effects of head injuries or brain trauma, such as concussions, and accelerating the healing thereof. Accordingly, in one exemplary embodiment, a composition is provided comprising components selected from a group including docosahexaenoic acid (DHA), curcumin and resveratrol. One benefit of the compositions disclosed herein is ingredient synergy, which may result in the reduction of the duration and/or severity of concussive symptoms and associated neurological damage from head trauma. Ingredient synergy may refer to the cooperative action of two or more ingredient components within a composition.

Specific advantages of the compositions disclosed herein may include a reduction in neuroinflammation, increased cerebral blood flow as well as a reduction in post-traumatic loss of neurons. As such, the compositions disclosed herein are useful for reducing the duration and/or severity of concussive symptoms and associated neurological damage from head trauma.

In one embodiment, a composition, e.g., a gel or slurry supplement, of easily absorbable ingredients is disclosed to be administered to or consumed by an individual entering a situation where the individual is likely to receive a blow to the head or for individuals who have recently suffered head trauma is disclosed. A preferred embodiment includes the composition being a gel or slurry supplement as discussed below. Other embodiments may include the composition having the form of a digestible pill, or a water-soluble powder.

The composition may have an effect of lessening the acute damage associated with head trauma. In addition, or in the alternative, the composition may assist in the neurological repair that typically follows head trauma. Specifically, one or more of the compositions disclosed herein may promote protection of the brain from oxidative stress following head trauma (e.g., concussions). Oxidative stress is known to occur as a result of concussions. Thus, the synergy of the ingredients comprising the composition may result in a reduction in the oxidative stress that follows a concussion. Additionally, or in the alternative, one or more compositions disclosed herein may stimulate the repair of nerve cells damaged as a result of head trauma. In one example, the intake of curcumin within a predetermined time period prior to the head trauma may reduce cerebral edema, which may develop as a result of inflammation caused by traumatic brain injury (TBI). Further, a composition including curcumin that is consumed within a predetermined time period prior to the head trauma may be capable of improving neurological function following TBI.

In one embodiment, one composition may include one or more of DHA, curcumin and resveratrol. For example, as will be discussed below in further detail, in one embodiment, the composition may include DHA and ingredients other than curcumin and resveratrol. In a second embodiment, the composition may include a composition of DHA, curcumin and ingredients other than resveratrol. In other embodiments, the composition may include DHA, resveratrol and ingredients other than curcumin. In another embodiment, the composition may include curcumin and ingredients other than DHA and resveratrol. In yet another embodiment, the composition may include curcumin, resveratrol and ingredients other than DHA. In still other embodiments, the composition may include resveratrol and ingredients other than DHA and curcumin. For example, in some embodiments, the composition may include one or more antioxidants and/or one or more branched-chain amino acids (BCAAs), such as leucine, isoleucine and valine. In one or more embodiments, the composition may include natural or artificial flavors or sweeteners. Examples of sweeteners may include, but are not limited or restricted to, honey, sugar or a sugar substitute (e.g., raw honey, sugar in the raw, Stevia, and coconut sugar).

As is well-understood, DHA, a primary structural component of the human brain, is an omega-3 fatty acid, and more specifically, is an omega-3 polyunsaturated fatty acid (n-3FA). DHA may be obtained through one's diet (e.g., via consumption of foods rich in omega-3 fatty acids such as salmon) or by the ingestion of eicosapentaenoic acid (EPA) in combination with docosapentaenoic acid (DPA). More specifically, omega-3 fatty acids include EPA, DHA and alpha-linolenic acid (ALA) among others. ALA is generally known as a metabolic precursor to both EPA and DHA, meaning ALA may be converted into EPA and DHA by the body. As is further understood, both DHA and EPA may be glycerides, including specifically, triglycerides. Thus, compositions disclosed herein may include multiple ingredients defined by a specific weight ratio of the ingredients. In other embodiments, some compositions disclosed herein may be defined by a ratio of the ingredients as measured in milligrams and/or mg/kg (milligrams of composition per kilogram of body weight of the individual consuming the composition).

In an exemplary embodiment, a method is provided for preventing traumatic brain injury, for example a concussion, wherein the method comprises administering or consuming a composition comprising at least a portion of DHA ranging from 300 mg and 500 mg, e.g., 350 mg. A portion of EPA may be included that ranges from 200 mg to 400 mg. Additionally, a portion of curcumin may be included that ranges from 50 mg/kg to 400 mg/kg. Further, a portion of resveratrol may be included that ranges from 10 mg to 100 mg.

In an exemplary embodiment, a composition for mitigating head trauma may be administered to an individual or consumed by an individual within a predetermined time period prior to a period wherein a traumatic head injury may be sustained (e.g., an activity or event having a high likelihood of resulting in an injury associated with head trauma, “the activity”). For example, one or more compositions disclosed herein may be administered or consumed within a one hour period prior to the activity. In other examples, one or more compositions disclosed herein may be administered or consumed within a two hour period, a three hour period, a twenty-four hour period, etc., prior to the activity. In one specific example, one or more compositions are administered or consumed within a 10 minute period prior to the activity. In yet other embodiments, one or more compositions are administered or consumed during the activity (e.g., during halftime of a sporting event, or after an individual's heart rate has been elevated above a predetermined threshold). Further, in other embodiments, one or more compositions are administered or consumed within a predetermined time period following the activity, e.g., when a traumatic brain injury occurred, such as a concussion. Additionally, one or more compositions are administered or consumed within a predetermined time period following the activity when it is undetermined or unlikely that traumatic brain injury occurred, e.g., following a sporting contest during which contact occurred with the individual's head (e.g., a soccer game during which a player routinely headed the soccer ball).

In some embodiments, compositions disclosed herein including DHA and EPA have a ratio of DHA to EPA comprising 2:1. In other embodiments, compositions disclosed herein including DHA and EPA have a ratio of DHA to EPA comprising 1:2, In yet other embodiments, compositions disclosed herein including DHA and EPA have a ratio of DHA to EPA being within the ratio ranges of 1:2 to 2:1. In alternative embodiments, compositions disclosed herein including DHA and EPA have a ratio of DHA to EPA comprising 3:2 or 2:3. As one specific embodiment, one composition disclosed herein including DHA and EPA includes 650 mg of omega-3 fatty acids including a ratio of DHA to EPA comprising 2:1, e.g., 433:217 mg.

In other embodiments, compositions disclosed herein including DHA, curcumin and resveratrol may include a ratio of DHA to resveratrol comprising 35:3. Exemplary amounts of the ingredients in such a combination may include 350 mg of DHA and 30 mg of resveratrol. Additionally, in some embodiments, the composition may include an amount of curcumin being within the range of 50-300 mg/kg, and in one particular example, 150 mg/kg.

### DETAILED DESCRIPTION

Embodiments of compositions including one or more of DHA, EPA, curcumin and resveratrol and methods for administering and/or consuming said compositions are described. One or more of the compositions provided may be capable of preventing and/or mitigating the effects of a traumatic brain injury (TBI), and specifically, preventing and/or mitigating the effects of oxidative damage resulting from TBI.

In the following description, certain terminology is used to describe various features of the invention. For example, the term “composition” generally refers to a combination of components or ingredients. As used herein, a composition may take any suitable form according to the included components or ingredients. Examples of forms that may be taken by compositions disclosed herein include, but are not limited or restricted to, a solid, a semi-solid, a gel, a slurry (e.g., a semiliquid mixture formed from solid, insoluble particles suspended in a liquid), a mist, a pill (solid, or an encapsulated liquid or gel). In yet other embodiments, one or more compositions disclosed herein may take the form of a transdermal patch that releases the composition through the skin. In such an embodiment wherein the composition has the form of a transdermal patch, the transdermal patch may be applied prior to an activity or event and release the composition prior to, during and, optionally, after the activity or event; thus, aiding in both the preventing and mitigation of concussive symptoms associated head trauma injuries.

In one or more of the compositions disclosed herein, each ingredient may be a natural product (e.g., generated by a living organism), or a plant or animal extract. Additionally, one or more ingredients of the one or more compositions disclosed herein may be synthesized using one or more chemical components. Each composition disclosed herein may utilize ingredient synergy, especially between DHA/EPA, resveratrol and/or curcumin in some embodiments, to provide nutritional building blocks and powerful antioxidants for the brain to potentially lessen the acute damage associated with head trauma, especially concussions. Further, the compositions disclosed herein may assist in the neurological repair following head trauma. In particular, the compositions disclosed herein, especially those containing at least DHA (or other omega-3 fatty acids), resveratrol and curcumin, may protect the brain from oxidative damage following trauma to the head as well as stimulate repair of nerve cells damaged during the incident causing trauma to the head.

As used herein and according to the Centers for Disease Control and Prevention (CDC), the term “concussion” may be defined as a type of traumatic brain injury (TBI) that is caused by a bump, blow or jolt to the head that causes the head and brain to move rapidly back and forth. The CDC further states that sudden movements, e.g., bumps, blows, or jolts to the head, can cause the brain to bounce around or twist in the skull, creating chemical changes in the brain, which may result in the damaging of brain cells.

Finally, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive. As this invention is susceptible to embodiments of many different forms, it is intended that the present disclosure is to be considered as an example of the principles of the invention and not intended to limit the invention to the specific embodiments shown and described.

### DHA/EPA, Curcumin, Resveratrol

Disclosed herein are compositions having ingredients that include one or more omega-3 fatty acids, resveratrol and/or curcumin. In some embodiments, compositions disclosed herein including DHA and resveratrol may have a ratio of DHA to resveratrol comprising 35:3. In some embodiments, compositions disclosed herein including DHA and resveratrol may have a ratio of DHA to resveratrol being within the ratio ranges of 15:1 to 4:1.

In some embodiments, compositions disclosed herein including EPA and resveratrol may have a ratio of EPA to resveratrol comprising 20:3. In other embodiments, compositions disclosed herein including EPA and resveratrol may have a ratio of EPA to resveratrol being within the ratio ranges of 9:1 to 2:1.

In yet other embodiments, compositions disclosed herein may include both DHA and EPA such that the compositions may include 650 mg of omega-3 fatty acids including a ratio of DHA to EPA comprising 2:1, e.g., 433:217 mg.

Further, compositions discussed above may include an amount of curcumin within a range of 50-200 mg/kg of curcumin. In one particular embodiment, one composition disclosed herein may include, for example, 150 mg/kg of curcumin.

### DHA/EPA, Curcumin

Disclosed herein are compositions having ingredients that include one or more omega-3 fatty acids and curcumin. In some embodiments, compositions disclosed herein including DHA and curcumin may have an amount of DHA comprising 350 mg and an amount of curcumin comprising 150 mg/kg. In some embodiments, compositions disclosed herein including DHA and curcumin may include an amount of DHA being within the range of 200-500 mg and an amount of curcumin being within the range of 50-200 mg/kg.

In some embodiments, compositions disclosed herein including EPA and curcumin may have an amount of EPA comprising 200 mg and an amount of curcumin comprising 150 mg/kg. In some embodiments, compositions disclosed herein including EPA and curcumin may include an amount of EPA being within the range of 100-400 mg and an amount of curcumin being within the range of 50-200 mg/kg.

### Curcumin, Resveratrol

Disclosed herein are compositions having ingredients that include curcumin and resveratrol. In some embodiments, compositions disclosed herein including curcumin and resveratrol may have an amount of curcumin comprising 150 mg/kg and an amount of resveratrol comprising 30 mg. In some embodiments, compositions disclosed herein including curcumin and resveratrol may include an amount of resveratrol being within the range of 5-50 mg and an amount of curcumin being within the range of 50-200 mg/kg.

### DHA/EPA, Curcumin, Resveratrol, α-Linoleic Acid

Like DHA and EPA, α-linoleic acid (ALA) is an omega-3 fatty acid. It is known that ALA cannot be produced by the human body; therefore, consumption through one's diet is critical. ALA can be found in many types of nuts and seeds such as, among others, chia seeds, flaxseeds, soybeans and oils such as linseed oil and canola oil. ALA plays an important role in maintaining DHA concentrations within the brain as ALA is a main precursor to the body's synthesis of DHA. Further, many studies have identified ALA as a nutraceutical due to its neuroplasticity, pleiotropic effects in neuroprotection, and vasodilation of brain arteries. These characteristics have resulted in many scientists and medical professions to link ALA consumption with stroke prevention.

Disclosed herein are compositions having ingredients that include one or more omega-3 fatty acids, resveratrol, ALA and/or curcumin. In some embodiments, compositions disclosed herein including DHA, resveratrol and ALA may have a ratio of DHA to resveratrol to ALA comprising 35:3:100. In some embodiments, compositions disclosed herein including DHA and resveratrol may have a ratio of DHA to resveratrol to ALA being within the ratio range of 15:1:50 to 4:1:20.

In some embodiments, compositions disclosed herein including EPA, resveratrol and ALA may have a ratio of EPA to resveratrol to ALA comprising 20:3:100. In other embodiments, compositions disclosed herein including EPA, resveratrol and ALA may have a ratio of EPA to resveratrol to ALA being within the ratio ranges of 9:1:50 to 2:1:20.

As mentioned above, in other embodiments, compositions disclosed herein may include both DHA and EPA such that the compositions may include 650 mg of omega-3 fatty acids including a ratio of DHA to EPA comprising 2:1, e.g., 433:217 mg. In addition to compositions including a ratio of DHA to EPA as disclosed herein, some such compositions may include resveratrol as well as ALA wherein a ratio of DHA to EPA to resveratrol to ALA may be 35:20:3:100. Additionally, compositions may include a ratio of DHA to EPA to resveratrol to ALA within the range of 15:9:1:50 to 4:2:1:20.

Further, as discussed above with respect to some embodiments, compositions including DHA and/or EPA, ALA and resveratrol may also include an amount of curcumin within a range of 50-200 mg/kg of curcumin. In one particular embodiment, one composition disclosed herein may include, for example, 150 mg/kg of curcumin.

### DHA/EPA, Curcumin, Resveratrol, Additional Ingredients

In some embodiments, compositions disclosed herein include varying ratios of DHA to resveratrol, EPA to resveratrol, and DHA to EPA to resveratrol as well as varying amounts of curcumin. In addition, some embodiments may include compositions comprising additional ingredients such as natural and/or artificial sweeteners or flavors, as referenced above. In yet further embodiments, compositions are disclosed that may include BCAAs to aid in protein synthesis and insulin production while also inhibiting the breakdown of muscle cells during and following an event or activity, e.g., a physical activity such as a sporting event.

In the foregoing description, the invention is described with reference to specific exemplary embodiments thereof. It will, however, be evident that various modifications and changes may be made thereto without departing from the broader spirit and scope of the invention as set forth in the appended claims.

What is claimed is:

1. A method for mitigating the effects of traumatic brain injury in an individual comprising:
administering an amount of a composition prior to a period wherein the traumatic brain injury is expected to occur, wherein the composition is comprised of:
300 mg and 500 milligrams (mg) of Docosahexaenoic acid (DHA),
50 mg/kg to 400 mg/kg (milligrams of composition per kilogram of body weight of the individual) of curcumin, and
10 mg to 100 mg of resveratrol.

2. The method of claim 1, wherein 350 mg of DHA, 150 mg/kg of curcumin and 30 mg of resveratrol is administered to the individual.

3. The method of claim 1, wherein the composition further includes α-linoleic acid.

4. The method of claim 3, wherein the composition includes 1-2 g of α-linoleic acid.

5. The method of claim 4, wherein the traumatic brain injury is a concussion.

6. The method of claim 4, wherein the composition is administered within a predetermined amount of time prior to a period wherein the traumatic brain injury is expected to occur.

7. The method of claim 6, wherein the predetermined amount of time is within an hour prior to the period wherein the traumatic brain injury is expected to occur.

8. The method of claim 6, wherein the predetermined amount of time is between one to three hours prior to the period wherein the traumatic brain injury is expected to occur.

9. The method of claim 4, wherein the composition is administered orally to the individual.

10. The method of claim 4, wherein the composition comprises a portion of an orally administered gel or slurry.

11. The method of claim 10, wherein the slurry is a semiliquid mixture formed from solid, insoluble particles suspended in a liquid

12. The method of claim 4, wherein the composition is administered to the individual during an onset of concussion symptoms.

13. The method of claim 4, wherein the composition is administered after an onset of concussion symptoms.
